(19) United States
(12) Patent Application Publication
(10) Pub. No.: US 2010/0203525 A1
(43) Pub. Date: Aug. 12, 2010

(54) DETECTION OF GENE DUPLICATIONS

(75) Inventors: Kenneth J. Livak, San Jose, CA (US); Junko Stevens, Menlo Park, CA (US); Katherine D. Lazaruk, Redwood City, CA (US); Janet S. Ziegle, Berkeley, CA (US); Lily Y. Wong, Oakland, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/614,318

(22) Filed: Nov. 6, 2009

Related U.S. Application Data
(63) Continuation of application No. 10/881,368, filed on Jun. 30, 2004, now abandoned.
(60) Provisional application No. 60/571,666, filed on May 14, 2004.

(51) Int. Cl. C12Q 1/68; G06F 19/00

(52) U.S. Cl. 435/6; 702/19

(57) ABSTRACT

Methods of detecting a candidate genetic anomaly such as a candidate duplication in a genome are disclosed. The methods comprise quantifying fluorogenic assays for alleles of a genetic locus from a plurality of individual genomes, identifying ranges of fluorescent intensities indicative of individual genomes homozygous for a first allele, homozygous for a second allele, or heterozygous for both alleles, and identifying individual genomes in which the fluorescence intensities are outside the range of intensities indicative of homozygosity or heterozygosity for the genetic locus.

### CROSS-REFERENCE TO RELATED APPLICATIONS

This application is a continuation of U.S. application Ser. No. 10/881,368, filed Jun. 30, 2004, which claims the benefit of U.S. Provisional Application No. 60/571,666, filed on May 14, 2004, which are both hereby incorporated by reference in their entireties.

### FIELD

This application relates generally to genetic analysis and, more particularly, to detection of genetic anomalies such as gene duplications.

### INTRODUCTION

The genetic complements of individuals of a species are known to vary. Genetic variations can occur at the scale of single nucleotide polymorphisms (SNPs) or at greater levels of organization. The latter can include, for example, genetic anomalies such as duplications, deletions, pseudogenes or rearrangements. Genetic anomalies can be causal of disease or influence disease severity or prognosis. Detection of genetic anomalies is thus of importance for clinicians and researchers However, analytical methods for analyzing the compositions of genomes do not always disclose candidate genetic anomalies.

### SUMMARY

Accordingly, the inventors have succeeded in devising new approaches to detecting a candidate genetic anomaly in a genome. This approach is based on polymerase chain reaction (PCR) assays, and, in particular, on fluorogenic assays which can be used to detect allelic differences such as SNPs. The methods, which utilize end-point fluorogenic PCR assays, can be used diagnostically and in genetic studies. Some of the candidate anomalies that can be detected are candidate gene duplications, candidate deletions, candidate pseudogenes, and candidate genetic rearrangements.

The inventors have also succeeded in devising new approaches for quantifying copy number of a DNA sequence in a genome. The DNA sequence can be any sequence comprised by a genome, such as a gene or a portion thereof. This approach is also based on polymerase chain reaction (PCR) assays, and can entail determining threshold detection cycle values in fluorogenic real time (RT) PCR assays. These approaches can be used for describing a genetic anomaly, for example for determining if a candidate genetic duplication is an actual genetic duplication. In various embodiments, the present teachings include methods of identifying a candidate genetic anomaly in a genome comprised by a population. The methods can comprise determining, in a fluorogenic assay for an allelic difference such as a SNP comprised by genomes of a plurality of individuals of the population, a range of fluorescence intensities of a first fluorophore indicative of a genome homozygous for a first allele of a genetic locus a range of fluorescence intensities of a second fluorophore indicative of a genome homozygous for a second allele of the genetic locus and a range of fluorescence intensities of the first and second fluorophores indicative of a genome heterozygous for the first and second alleles of the genetic locus, and determining if the fluorescence intensities of the first and the second fluorophores of the fluorogenic assay for the allele in a genome of one or more individuals of the population are outside the ranges of fluorescence intensities indicative of a genome that is homozygous for the first allele, homozygous for the second allele, or heterozygous for the first and second alleles.

In various embodiments, a detection method can utilize any probe which can detect a nucleic acid sequence quantifiably. In some configurations, a detection probe can be, for example, a 5′-exonuclease assay probe such as a TaqMan® probes described herein, various stem-loop molecular beacons, stemless or linear beacons, PNA Molecular Beacons™, linear PNA beacons, non-FRET probes, Sunrise®/Amplifluor® probes, stem-loop and duplex Scorpion™ probes, bulge loop probes, pseudo knot probes, cyclicons, MGB Eclipse™ probe (Epoch Biosciences), hairpin probes, peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Labeling probes can also comprise black hole quenchers (Biosearch), Iowa Black (IDT), QSY quencher (Molecular Probes), and Dabsyl and Dabcel sulfonate/carboxylate Quenchers (Epoch). Labeling probes can also comprise sulfonate derivatives of fluorescent dyes, phosphoramidite forms of fluorescein, or phosphoramidite forms of CY5. In some embodiments, interchelating labels can be used such as ethidium bromide, SYBR® Green I, and PicoGreen®, thereby allowing visualization in real-time, or end point, of an amplification product in the absence of a labeling probe.

In various configurations, a detection probe can comprise a fluorophore and a fluorescence quencher. The detection probe, in these embodiments, can be used in a 5′ nuclease assay such as a fluorogenic 5′ nuclease assay, such as a Taqman® assay, in which the fluorophore or the fluorescence quencher is released from the detection probe if the detection probe is hybridized to the detection probe hybridization sequence. In these embodiments, the 5′ nuclease assay can utilize 5′ nucleolytic activity of a DNA polymerase that catalyzes a PCR amplification of a probe set ligation sequence. The fluorogenic 5′ nuclease detection assay can be a real-time PCR assay or an end-point PCR assay. The fluorophore comprised by a detection probe in these embodiments can be any fluorophore that can be tagged to a nucleic acid, such as, for example, FAM, VIC, Sybra Green, TET, HEX, JOE, NED, LIZ, TAMRA, ROX, ALEXA, Texas Red, Cy3, Cy5, Cy7, Cy9, or dR6G.

In certain embodiments, fluorescence intensity can be measured as an end point fluorescence intensity value. In some aspects, an end point fluorescence intensity value can be an intensity value normalized to a standard, such as a fluorophore of known fluorescence intensity.

In other embodiments, the present teachings include methods of identifying a candidate genetic anomaly in the genome of an individual of a population. The method can comprise determining, in a fluorogenic assay for a genetic locus comprised by genomes of a plurality of individuals of the species, a range of fluorescence intensities of a first fluorophore which are indicative of a genome homozygous for a first allele, a range of fluorescence intensities of a second fluorophore indicative of a genome homozygous for a second allele, and a range of fluorescence intensities of the first and second fluorophores indicative of a genome heterozygous for the first and second alleles. In these embodiments, a fluorogenic assay for alleles can also be performed on a genome of an individual, such as a human patient. The fluorescence intensities of the first and the second fluorophores of the fluorogenic assay for an allele of a genetic locus of the individual's genome can be determined to be either within or outside the ranges of fluorescence intensities indicative of homozygosity or heterozygosity for either allele. If a fluorescence intensity of either or both fluorophores is outside the range of fluorescence intensities indicative of a genome homozygous or heterozygous for the allele, the sample can be considered to comprise a candidate genetic anomaly. Methods for performing allelic classification and genotyping can involve developing likelihood models such as those disclosed in U.S. patent application Ser. No. 10/611,414 of Holden et al., filed Jun. 30, 2003, which is incorporated by reference in its entirety herein. In some configurations, these methods can comprise evaluating fluorescence intensity data for each of the plurality of individuals to identify one or more data ranges or clusters, each range or cluster associated with a discrete allelic combination, and generating a likelihood model that predicts the probability that a selected sample will reside within a particular data range or cluster based upon its intensity information. In these embodiments, a cluster can comprise at least two data points. The methods can further comprise applying the likelihood model to each of the plurality of samples to identify individuals not residing an any range or cluster, and identifying one or more of these individuals as comprising a candidate genetic anomaly such as a gene duplication.

In yet other embodiments, the present teachings include a system for identifying a candidate genetic anomaly in a population. In these embodiments, the system can comprise a graphical interface. A graphical user interface can be comprised by a digital computer monitor. In various configurations, a graphical user interface can exhibit a plurality of data points, wherein each data point occupies a position representing fluorescence intensities of a first fluorophore and a second fluorophore from an individual genomic sample subjected to a fluorogenic assay for an allele of a genetic locus, wherein fluorescence intensity of a first fluorophore can be indicative of the presence of a first allele and fluorescence intensity of a second fluorophore can be indicative of the presence of a second allele, and wherein a cluster of data points can be indicative of a genome homozygous for the first allele, a genome homozygous for the second allele, or a genome heterozygous for the first and second alleles. In certain configurations, the graphical interface can comprise a scatterplot for displaying the data. A display of a scatter plot can include coordinate axes, such as orthogonal coordinate axes. A display In these embodiments, a cluster can comprise at least two data points. In various configurations, a data point outside any cluster can represent an individual genome comprising a candidate genetic anomaly.

In additional embodiments, the present teachings include methods of identifying a candidate genetic anomaly in the genome of a subject individual using a graphical interface. The method can comprise exhibiting in a graphical interface a plurality of data points, wherein each data point occupies a position representing fluorescence intensities of a first fluorophore and a second fluorophore from a genomic sample of a reference population subjected to a fluorogenic assay for alleles of a genetic locus, wherein fluorescence of a first fluorophore can be indicative of the presence of a first allele and fluorescence of a second fluorophore can be indicative of the presence of a second allele. In these embodiments, a cluster of data points can be indicative of a genome homozygous for the first allele, a genome homozygous for the second allele, or a genome heterozygous for the first and second alleles. A data point which occupies a position representing fluorescence intensities of the first fluorophore and the second fluorophore from fluorogenic SNP assays of a subject individual can also be exhibited in the graphical interface. A determination that the data point from the test individual falls outside any cluster indicates that the individual's genome comprises a candidate genetic anomaly. In certain configurations, fluorescence intensity can be measured as a threshold cycle (C_{T}) value in a real time polymerase chain reaction coupled with a fluorogenic assay. In certain other configurations, fluorescence intensity can be measured as an end point value. An end point fluorescence intensity value can be an intensity value measured directly, or can be a value normalized to a standard, such as a fluorophore of known fluorescence intensity.

In the above embodiments, a fluorogenic assay can comprise a mixture comprising a genome of an individual; a first nucleobase primer comprising a sequence which maps upstream from a genetic locus; a second primer complementary to a sequence which maps downstream from the genetic locus; a first probe comprising the first fluorophore, and a nucleic acid sequence complementary to a genomic sequence comprising the first allele; and a second probe comprising a second fluorophore, the fluorescence quencher, and a nucleic acid sequence complementary to a genomic sequence comprising the second allele; and a DNA polymerase. In various configurations, the DNA polymerase can be a thermostable DNA polymerase such as a taq polymerase, and the hybridization conditions can be high stringency conditions. In these configurations, the mixture can be subjected to thermal cycling, which can lead to sequence amplification by a polymerase chain reaction (PCR). In these configurations, fluorescence of the first or second fluorophores a can be detected as an end point, i.e., following a fixed number of cycles in a polymerase chain reaction. In various configurations, each of the upstream primer and the downstream primer can comprise from at least about 10 nucleotides up to about 70 nucleotides, or from about 15 nucleotides up to about 40 nucleotides, or from about 20 nucleotides up to about 30 nucleotides. In various configurations, each SNP probe can comprise from at least about 10 nucleotides up to about 50 nucleotides, from at least about 12 nucleotides up to about 25 nucleotides, or from at least about 13 nucleotides up to about 18 nucleotides. In certain configurations, probes comprising a minor groove binder can comprise from at least about 13 up to about 18 nucleotides.

The mixture described above can further comprise a control fluorophore. A control fluorophore can provide a uniform control fluorescence signal. Fluorescence resulting from digestion of a probe can be quantified by measuring its fluorescence intensity relative to that of the control fluorophore. Hence, in some configurations, fluorescence intensity of a fluorophore, such as a fluorophore released from inhibition of can be normalized to the fluorescence intensity of the control fluorophore. In these configurations, fluorescence intensity of a fluorophore can be expressed as a unitless ratio of fluorescence intensity of a fluorophore released from a probe compared to that of control fluorophore. In some configurations, each of the normalized fluorescence intensities within a range differ from at least one other normalized fluorescence intensity within the range by no more than about 20%, no more than about 15%, no more than about 10%, no more than about 5%, or no more than about 2%.

Candidate genetic anomalies which can be revealed in some configurations of the methods described herein can include, for example, potential extra copies of sequences such as gene duplications; potential pseudogenes; potential additional alleles; potential deletions; potential additional SNPs comprised by a sequence complementary to a probe; or potential additional SNPs comprised by a sequence complementary to a primer.

In various configurations of the present teachings, a first fluorophore and a second fluorophore comprised by probes used to detect the alleles in a fluorogenic assay can be different, and can be selected from many different fluorophores. These fluorophores can be, for example, commercially available fluorophores such as FAM, VIC, Sybra Green, TET, HEX, JOE, NED, LIZ, TAMRA, ROX, ALEXA, Texas Red, Cy3, Cy5, Cy7, Cy9, and dR6G. Among these fluorophores, FAM and VIC can be used effectively in paired probes, in which each probe hybridizes to a different SNP allele. In addition, in some configurations, a fluorophore that does not comprise a SNP probe can be used as a control fluorophore. For example, the fluorophore ROX can be used as a control fluorophore in a SNP assay using a first probe comprising a FAM fluorophore and a second probe comprising a VIC fluorophore.

In various embodiments of the present teachings, the above described methods for identifying candidate genetic anomaly can be used to identify genetic anomalies such as, for example, gene duplications in genes of clinical or research interest. Some genes of potential clinical or research interest for which candidate genetic lesions can be identified using the above described methods include, for example, a cytochrome P450 gene such as a CYP1A1 gene, a CYP1A2 gene, a CYP2A1 gene, a CYP2A6 gene, a CYP2A7 gene, a CYP2B6 gene, a CYP2C8 gene, a CYP2C9 gene, a CYP2C19 gene, a CYP2D6 gene, a CYP2E1 gene, a CYP3A4 gene, a CYP3A5 gene, a CYP3A7 gene, a CYP4B1 gene, a CYP5A1 gene, a CYP8A1 gene, or a CYP21 gene; .a NAT1 gene, a NAT2 gene, a COMT gene, a TMPT gene, a TYMS gene, a constitutive androstane receptor gene, a pregnane X receptor gene, an alcohol dehydrogenase gene, a flavin monooxygenase gene, a glutathione S-transferase gene, a transporter gene, an oATP-C gene, an epoxide hydrolase gene, a carboxylesterase gene, a monoamine oxidase gene, a paraoxonase gene, sulfotransferase gene, a UDP-glucuronosyl-transferase gene, an ADH1A gene, an ADH1B gene, an ADH1C gene, an ADH4 gene, an ADH5 gene, an ADH6 gene, an ADH7 gene, an FM01 gene, an FM03 gene, an FM04 gene, an FM05 gene, a GSTM1 gene, a GSTT1 gene, a multidrug resistance gene such as an MDR1 gene, an MRP1 gene, an MRP2 gene, and an MXR gene. Hence, in certain configurations, the above described techniques for identifying candidate genetic anomalies can be used diagnostically.

Various embodiments of the present teachings include methods of determining a copy number of the target gene in a sample genome. These methods can comprise forming a reaction mixture which comprises a sample comprising the sample genome, a target sequence primer pair, a target sequence detection probe comprising a first fluorophore, an endogenous reference sequence primer pair, an endogenous reference sequence detection probe comprising a second fluorophore, and a DNA polymerase such as a thermostable polymerase such as taq polymerase. In these embodiments the first fluorophore and the second fluorophore can be different, and each can be selected from the group consisting of FAM, VIC, Sybra Green, TET, HEX, JOE, NED, LIZ, TAMRA, ROX, ALEXA, Texas Red, Cy3, Cy5, Cy7, Cy9, and dR6G. In some configurations, the first fluorophore and the second fluorophore can be selected from FAM and VIC.

In various configurations, a calibrator reaction mixture which comprises a calibrator sample of known copy number of the target sequence and known copy number the reference sequence can also be formed. The calibrator reaction mixture can also comprise the target sequence primer pair, the target sequence detection probe comprising the first fluorophore, the endogenous reference sequence primer pair, the endogenous reference sequence detection probe comprising a second fluorophore, and the DNA polymerase such as a thermostable polymerase such as taq polymerase. The target sequence and the reference sequence comprised by the sample can be amplified by thermal cycling using real time detection in a polymerase chain reaction. Threshold cycle values for detection of the target sequence and the endogenous reference sequence can be determined for the sample genome. In some configurations, threshold cycle values for detection of the target sequence and the endogenous reference sequence can be determined for the calibrator sample. From the threshold cycle values, the amount of target sequence normalized to the reference sequence and relative to the calibrator can be determined. The amount of target normalized target sequence can be expressed as a ratio of copy number of the target sequence to the copy number of the reference sequence in the sample genome. If the copy number of the reference sequence is known, copy number of the target sequence can be calculated. In some configurations, ratios can be adjusted for experimental variations, for example by rounding off the ratio to the nearest whole number.

In various configurations, the target sequence detection probe and the endogenous reference sequence detection probe can each further comprise a minor groove binder. Furthermore, the target sequence detection probe and the endogenous reference sequence detection probe can each comprise from about 10 nucleotides to about 50 nucleotides, from about 12 nucleotides to about 25 nucleotides, or from about 13 nucleotides to about 18 nucleotides.

In various configurations of these embodiments, determining the amount of target sequence, normalized to the reference sequence and relative to a calibrator, can comprise determining −ΔΔC_{T}, wherein C_{T} is the threshold number of cycles for detection of a fluorophore in a real time PCR assay; C_{T,q} is the threshold number of cycles for detection of a fluorophore for the target sample in the real time PCR assay, C_{T,cb} is the threshold number of cycles for detection of a fluorophore for a calibrator sample in the real time PCR assay, ΔC_{T, q} is a difference in threshold cycles for the target and the endogenous reference, ΔC_{T, cb} is a difference in threshold cycles for the calibrator sample and the endogenous reference, and −ΔΔC_{T}=ΔC_{T,q}−ΔC_{T,cb}. In these configurations, if −ΔΔC_{T} is determined, the relative quantity of the target sequence can be determined using the relationship that relative quantity can be equal to 2^{-ΔΔCT}. In some configurations, the copy number of the target gene in the sample can determined by multiplying the relative quantity by the number of copies of the endogenous reference sequence. In some configurations, the endogenous reference sequence can be a single copy gene in a haploid genome, i.e., a two-copy gene in a diploid genome. The reference sequence, in some configurations, can be a two-copy sequence in a diploid genome such as, for example, an RNase P gene.

In various configurations, the target sequence can be, for example, a sequence comprised by a gene in which copy number can be of clinical relevance. In some configurations, the target sequence can be comprised by a gene selected from the group consisting of a cytochrome P450 gene such as a CYP1A1 gene, a CYP1A2 gene, a CYP2A1 gene, a CYP2A6 gene, a CYP2A7 gene, a CYP2B6 gene, a CYP2C8 gene, a CYP2C9 gene, a CYP2C19 gene, a CYP2D6 gene, a CYP2E1 gene, a CYP3A4 gene, a CYP3A5 gene, a CYP3A7 gene, a CYP4B1 gene, a CYP5A1 gene, a CYP8A1 gene, or a CYP21 gene; .a NAT1 gene, a NAT2 gene, a COMT gene, a TMPT gene, a TYMS gene, a constitutive androstane receptor gene, a pregnane X receptor gene, an alcohol dehydrogenase gene, a flavin monooxygenase gene, a glutathione S-transferase gene, a transporter gene, an oATP-C gene, an epoxide hydrolase gene, a carboxylesterase gene, a monoamine oxidase gene, a paraoxonase gene, sulfotransferase gene, a UDP-glucuronosyl-transferase gene, an ADH1A gene, an ADH1B gene, an ADH1C gene, an ADH4 gene, an ADH5 gene, an ADH6 gene, an ADH7 gene, an FM01 gene, an FM03 gene, an FM04 gene, an FM05 gene, a GSTM1 gene, a GSTT1 gene, an MDR1 gene, an MRP1 gene, an MRP2 gene, and an MXR gene.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is an illustration of data describing discrimination of alleles for SNP using BIC-NFQ-MGB and FMA-NFQ-MGB probes on genomic DNA samples from human individuals.

FIG. 2 is an illustration of a data set from a CYP2D6 gene dosage assay of 31 individuals.

FIG. 3 is an illustration of a data set from a GSTM1 gene dosage assay of 31 individuals.

FIG. 4 is an illustration of multiclustered patterns in CYP2E1 assays of genomic DNA.

FIG. 5 is an illustration of data sets from CYP2E1 gene dosage assays of 28 individuals.

FIG. 6 is an illustration of primer and probe selection for a SNP from human gene CYP2B6.

FIG. 7 is a diagram of a TaqMan® fluorogenic 5′ nuclease SNP assay.

FIG. 8 is a table of gene dosage assays to CYP2E1.

### DETAILED DESCRIPTION

Methods and apparatus for the detection of gene duplication are described herein. The methods and apparatus described herein utilize laboratory techniques well known to skilled artisans and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999.

Real time and end point detection of amplification by PCR, can be conducted using methods well known in the art. In various embodiments, a detection methods can utilize any probe which can detect a nucleic acid sequence. In some embodiments, a detection method can comprise a fluorescence assay in which a fluorescence signal can be detected that can be indicative of probe binding to its target. In some configurations, a detection probe can be, for example, a fluorogenic 5′-exonuclease assay probe such as a TaqMan® probe described herein, various stem-loop molecular beacons, stemless or linear beacons, PNA Molecular Beacons™, linear PNA beacons, non-FRET probes, Sunrise®/Amplifluor® probes, stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Labeling probes can also comprise black hole quenchers (Biosearch), Iowa Black (IDT), QSY quencher (Molecular Probes), and Dabsyl and Dabcel sulfonate/carboxylate Quenchers (Epoch). Labeling probes can also comprise two probes, wherein for example a flore is on one probe, and a quencher on the other, wherein hybridization of the two probes together on a target quenches the signal, or wherein hybridization on target alters the signal signature via a change in florescence. Labeling probes can also comprise sulfonate derivatives of fluorescenin dyes with SO3 instead of the carboxylate group, phosphoramidite forms of fluorescein, phosphoramidite forms of CY 5 (available for example from Amersham). In some embodiments, interchelating labels can be used such as ethidium bromide, SYBR® Green I (Molecular Probes), and PicoGreen® (Molecular Probes), thereby allowing visualization in real-time, or end point, of an amplification product in the absence of a labeling probe.

A detection probe in some embodiments can be a Taqman® probe, and, in some configurations, can comprise a fluorophore and a fluorescence quencher, for example as described in Lee, L. G., et al. Nucl. Acids Res. 21:3761 (1993), and Livak, K. J., et al. PCR Methods and Applications 4: 357 (1995). The fluorescence quencher can be a fluorescent fluorescence quencher, such as the fluorophore TAMRA, or a non-fluorescent fluorescence quencher (NFQ), for example, a combined NFQ-minor groove binder (MGB) such as an MGB Eclipse™ minor groove binder supplied by Epoch Biosciences (Bothell, Wash.) and comprised by TaqMan® probes (Applied Biosystems, Inc.) The fluorophore can be any fluorophore that can be attached to a nucleic acid, such as, for example, FAM, VIC, Sybra Green, TET, HEX, JOE, NED, LIZ, TAMRA, ROX, ALEXA, Texas Red, Cy3, Cy5, Cy7, Cy9, or dR6G. Methods for detecting fluorescence from enzymatic hydrolysis of a fluorogenic probe such as a TaqMan® probe are well known in the art. Upon hybridization of PCR primers and detection probe to a probe set ligation sequence, a DNA polymerase comprising a detection mixture can catalyze hydrolysis of the probe, for example during thermal cycling, and thereby release the fluorophore from inhibition of its fluorescence by the fluorescence quencher. A resulting increase in fluorescence of a fluorophore released from quenching by 5′ nuclease digestion of a fluorogenic probe can be indicative of the presence of a small RNA in a sample. For example, TaqMan® probes and primers, and taq polymerase, distributed by Applied Biosystems, Inc., can be utilized in the methods of certain configurations described herein. The TaqMan® probes can be, in some configurations, pairs of probes, each probe directed to one allele of a SNP. In certain other configurations, TaqMan® probes can be directed to alleles of a genomic sequence larger than a SNP.

Various embodiments of the present teachings comprise methods of identifying a candidate genetic anomaly in genomes of a population. These methods can comprise determining, in a fluorogenic assay for alleles of a genetic locus comprised by genomes of a plurality of individuals of the population, a range of fluorescence intensities of a first fluorophore indicative of a genome homozygous for a first allele, a range of fluorescence intensities of a second fluorophore indicative of a genome homozygous for a second allele, and a range of fluorescence intensities of the first and second fluorophores indicative of a genome heterozygous for the first and second alleles, and determining if the fluorescence intensities of the first and the second fluorophores of the fluorogenic assay of one or more individuals of the species are outside the ranges of fluorescence intensities indicative of a genome that can be homozygous for the first allele, homozygous for the second allele, or heterozygous for the first and second alleles. A fluorogenic assay can be, in non-limiting example, a TaqMan® assay. Fluorescence intensities can be measured using standard laboratory equipment, such as, for example, an ABI Prism 7700 Sequence Detection System (Applied Biosystems, Inc.) Such systems can facilitate the collection and display of data generated in real time or end point detection PCR. Methods for conducting PCR assays for allele detection are well known in the art, and are discussed in detail in publications such as “Allelic discrimination using the 5′ nuclease assay,” Applied Biosystems 2001.

In various embodiments, amplification of an endogenous control can be performed to standardize the amount of sample RNA or DNA added to a reaction. Relative quantification can be performed using a standard curve method or a comparative method. The following delimitations are assumed in this description of relative quantification.

“Standard” as used herein is a sample of known concentration used to construct a standard curve. “Reference” as used herein can be a passive or active signal used to normalize experimental results. Endogenous and exogenous controls can be examples of active references. Active reference can refer to a signal generated as the result of PCR amplification. The active reference can have its own set of primers and probe. “Endogenous control” as used herein can be a nucleic acid that can be present in each experimental sample as isolated. “Exogenous control” as used herein can be a nucleic acid added (or “spiked”) into each sample to a known concentration. An exogenous active reference can be, for example, an in vitro construct that can be used as an internal positive control (IPC) to distinguish true target negatives from PCR inhibition. An exogenous reference can also be used, for example, to normalize for differences in efficiency of sample extraction. A passive reference can be, for example, a control fluorophore such as, for example, the dye ROX. A control fluorophore can be used to normalize for non-PCR related fluctuations in fluorescence signal. “Calibrator,” as used herein, can be a sample used as the basis for comparative results. A calibrator can be, for example, a sample genome comprising a known copy number of a target sequence, a known copy of a reference sequence, or both.

Various embodiments of the present teachings describe methods of identifying a candidate genetic anomaly in genomes of a population. These methods can comprise determining, in a fluorogenic assay for alleles of a genetic locus comprised by genomes of a plurality of individuals of the population, a range of fluorescence intensities of a first fluorophore indicative of a genome homozygous for a first allele, a range of fluorescence intensities of a second fluorophore indicative of a genome homozygous for a second allele, and a range of fluorescence intensities of the first and second fluorophores indicative of a genome heterozygous for the first and second alleles of the genetic locus, and determining if the fluorescence intensities of the first and the second fluorophores of the fluorogenic assay for an allele of the genetic locus of a genome of one or more individuals of the species are outside the ranges of fluorescence intensities indicative of a genome that can be homozygous for the first SNP allele, homozygous for the second SNP allele, or heterozygous for the first and second SNP alleles. In these embodiments, the determination of ranges can involve comparing fluorescence intensities. Fluorescence intensity can be reported as the ratio of the fluorescence of the fluorophore to that of a control fluorophore such as ROX. In some configurations, when differences in intensities between individuals can be as small as 2%, as small as 5%, as small as 10%, as small as 15%, or as small as 20%, these individuals can be considered to share the same genotype. Hence, in various embodiments, fluorescence intensities falling into a range can be considered to represent individuals sharing a genotype. In certain other configurations, statistical analytical methods, such as those set forth in U.S. patent application Ser. No. 10/611,414, can be applied to identify clusters of fluorescence intensities indicative for genomes homozygous or heterozygous for a genetic locus within a population. In addition, analytical methods set forth in U.S. patent application Ser. No. 10/611,414 can also be used to identify outliers, i.e., individuals that do not fall into a cluster. In some configurations of the present teachings, outliers can represent candidate genetic anomalies such as candidate gene duplications.

In some configurations, a genetic anomaly can be a genetic anomaly of a target sequence which can be a haploid sequence, such as, for example, a gene comprised by a Y chromosome in a mammal. In such cases, only two ranges or two clusters are expected for hemizygous genetic loci from individuals without a genetic anomaly in the target sequence. Haploid sequences can otherwise be analyzed similarly to diploid sequences.

In various embodiments, for some individuals, fluorescence intensities can be more intense for the first fluorophore (which can be, for example, FAM) compared to the second fluorophore (which can be, for example, VIC). These individuals can be considered homozygous for a first allele being assayed. Similarly, for some individuals, fluorescence intensities will be more intense for the second fluorophore compared to the first fluorophore. These individuals can be considered homozygous for a second allele being assayed. In addition, samples which exhibit intermediate intensities of both fluorophores can be from individuals heterozygous for the SNP. However, some individuals will exhibit fluorescence intensities that fall outside the ranges for either a homozygous or heterozygous allelic profile. The normalized fluorescence intensities for these individuals can, for example, differ by at least about 2%, at least about 5%, at least about 10%, at least about 15%, or at least about 20% from individuals assigned to a range corresponding to homozygous or heterozygous individual. These “outliers” can be considered to be comprising a candidate genetic anomaly. In some configurations, allelic analyses can further include no template controls (NTC's). In certain alternative embodiments, the experimental data can be analyzed using likelihood models disclosed in U.S. patent application Ser. No. 10/611,414 of Holden. However, in certain configurations, outliers revealed in such analyses can be considered to represent genetic anomalies such as gene duplications.

In various embodiments, a candidate genetic anomaly can be, in non-limiting example, a candidate gene duplication, a candidate supernumerary allele or a candidate pseudogene. Other phenomena that can potentially lead to detection of outliers include the presence of an allele , for example, a previously unreported allele within a sequence covered by a primer in the fluorogenic assay, or a previous unreported allele within a sequence covered by the primers in the assay.

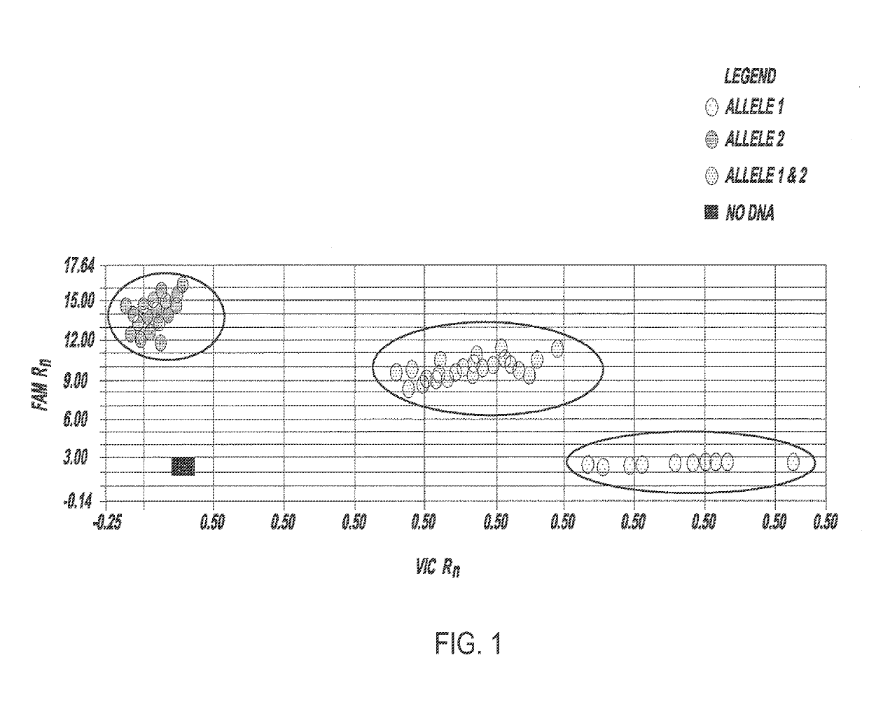

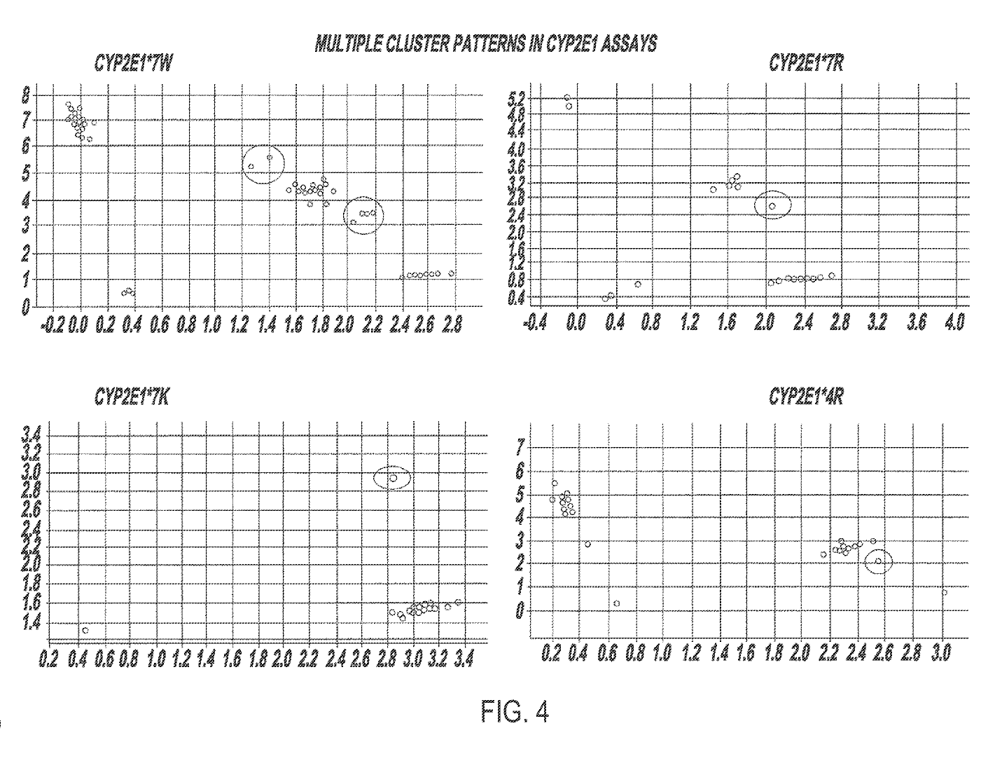

In various embodiments, the data generated from such assays can be analyzed with the aid of a graphical display. In various embodiments, graphic representation can comprise a Cartesian coordinate system wherein position each axis can be indicative of fluorescence intensities of the fluorophores. For example, the vertical (Y) axis can be representative of normalized FAM fluorescence intensity, while the horizontal (X) axis can be representative of normalized VIC fluorescence intensity. If normalized fluorescence intensities for a plurality of individuals are plotted on such a display, a graphic representation of the data can be provided. A graphical display can comprise a manually generated display, such as a hand-drawn graph on graph paper, or an automated graphical display, such as, for example, a display generated using graphing software and a digital computer. In most cases, the majority of the combined data points from a plurality of individuals will fall into three clusters, representing individuals homozygous for a first allele, individuals homozygous for a second allele, and individuals heterozygous for both alleles (see, for example, FIG. 1). However simple visual inspection can reveal the presence of one or more “outliers” in a graphical representation (see, for example, the circled data points in FIG. 4 graph labeled CYP2E1*7). These individual genomes can be identified as comprising a candidate genetic anomaly. A Cartesian coordinate system can further comprise, in some embodiments, an origin representing zero fluorescence.

In various embodiments, the inclusion or exclusion of a data point in a cluster can be based on the criteria of absolute numerical differences in fluorescence intensity, as discussed supra regarding determination of ranges of fluorescence intensities. However, in some configurations, additional criteria can also be applied. First, it is expected that data points from individuals homozygous for alleles will form clusters near the axes and heterozygotes will form a cluster at an intermediate position, and, in various configurations, that genetic anomalies such as gene duplications can be expected to provide data points which will fall between the clusters. Hence, in certain aspects, a data point that is very far removed from any cluster can be considered to be an artifact. In some configurations, an outlier that is more than about 40%, more than about 50% or more than about 60% of the distance from any data point within a cluster (compared to the distance between the cluster data point and the origin or the distance between the cluster data point and no template control data points, can be considered to be an artifact, for which further analysis is not indicated.

Another criterion that can be applied in assessing whether an outlier data point represents a candidate genetic anomaly can be position of the data point with respect to a cluster of homozygous or heterozygous individuals, and a cluster of data points representing no template controls. In certain configurations, it can be expected that an outlier data point which can be positioned in an approximate straight line between a cluster of homozygous or heterozygous individuals and the no template controls can represent an individual that belongs in the cluster, but for unknown reasons the assay was not performed efficiently. For example, if the sample was degraded or contaminated, the assays might have proceeded with low efficiency.

Yet another criterion that can be applied in assessing whether an outlier data point represents a candidate genetic anomaly can be whether assays for other alleles on the same individual genome also result in the appearance of outliers. For example, if additional assays were conducted using additional alleles that map within the same gene or haplotype, or, for example, within about 100 kilobases of the genetic locus providing an outlier data point, outlying data points from these alleles could be used to designate the individual as comprising a genetic anomaly.

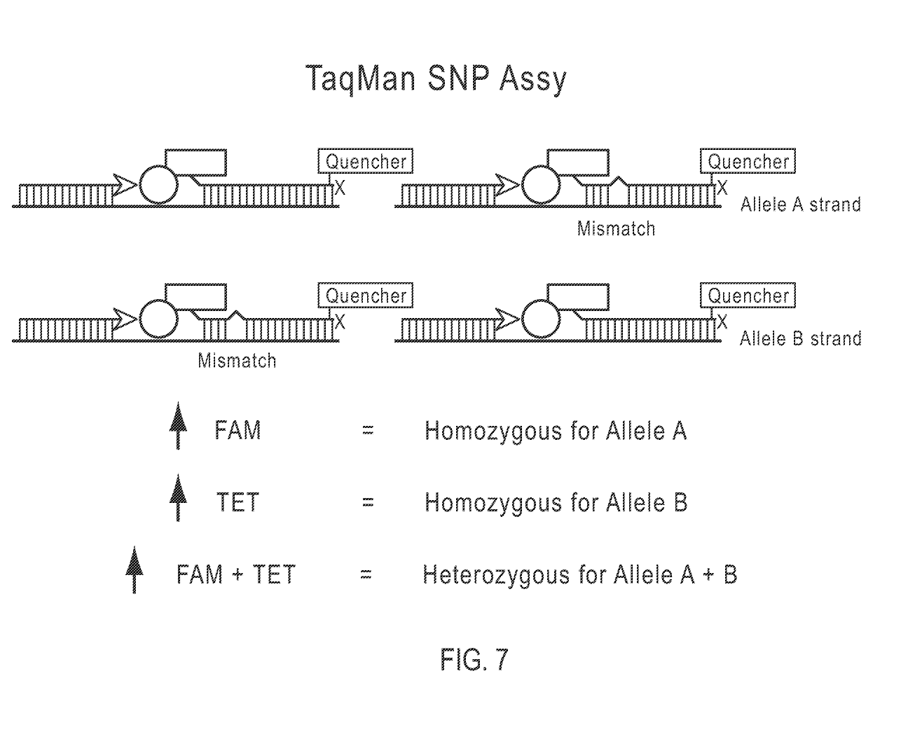

In various embodiments, anomalies that can be revealed after initially identifying candidate genetic anomalies using the methodology disclosed above, can include, for example, extra copies of a gene; the presence of a third allele (see, for example, FIG. 7; non-specific primers; an allele under a primer; a second allele under a probe which causes the appearance of a supernumerary “angle cluster,” i.e., one that does not lie within an approximate straight line between a cluster and the no template controls; or a second allele under the probe which causes a supernumerary “vector cluster” which lies in an approximate straight line between a cluster and the no template controls.

Certain embodiments of the present teachings involve methods of relative quantification of DNA sequences. Some related quantification techniques are discussed in discussed in detail in the publication “User Bulletin #2 ABI Prism 7700 Sequence Detection System,” Applied Biosystems, 2001. This publication is available for download on the internet at www.appliedbiosystems.com. This publication is incorporated herein by reference in its entirety.

Certain configurations of some embodiments of the present teachings can involve reference to a standard curve. The preparation of a standard curve is well known in the art. In some configurations, target quantity can be determined from a standard curve and normalized to the target quantity of the calibrator. Thus, the calibrator can be considered the 1× sample, and other quantities can be expressed as in an N-fold difference relative to the calibrator. Because in some configurations the sample quantity can be divided by the calibrator quantity, a sample quantity can be expressed as a unitless ratio. Accordingly, for relative quantification, any stock RNA or DNA containing the appropriate target can be used to prepare standards. In some configurations, a calibrator sample can be used as a standard.

In some configurations, comparative C_{T} methods can be used to analyze sequence copy number. These methods are similar to the standard curve methods, except that arithmetic formulas are used to achieve the same result for relative quantification. In these methods, the amount of target, normalized to an endogenous reference and relative to a calibrator, is given by:

2^{-ΔΔCT}

The derivation of the above formula can be as follows. The equation that describes the exponential amplification of PCR is:

Xₙ=X_{O}×(1+E_{X})ⁿ

wherein:
Xₙ=number of target molecules at cycle n
Xₒ=initial number of target molecules
E_{X}=efficiency of target amplification
n=number of cycles

The threshold cycle (C_{T}) indicates the fractional cycle number at which the amount of amplified target reaches a fixed threshold. Thus,

X_{T}=Xₒ×(1+Eₓ)C_{T,x}=K_{X}

where:
X_{T}=threshold number of target molecules
C_{T,X}=threshold cycle for target amplification
K_{X}=constant

A similar equation for the endogenous reference reaction is:

R_{T}=R_{O}×(1+E_{R})C_{T,R}=K_{R}

where:
R_{T}=threshold number of reference molecules
R_{O}=initial number of reference molecules
E_{R}=efficiency of reference amplification
C_{T,R}=threshold cycle for reference amplification
K_{R}=constant Dividing X_{T} by R_{T} gives the following expression:

$$\frac{{X}_{T}}{{R}_{T}} = \frac{{X}_{O} {x ( 1 = {E}_{x} )}^{{C}_{T , X}}}{{R}_{O} {x ( 1 + {E}_{R} )}^{{C}_{T , R}}} = \frac{{K}_{X}}{{K}_{R}} = K$$

The exact values of X_{T} and R_{T} can depend on a number of factors, such as: reporter dye used in the probe, sequence context effects on the fluorescence properties of the probe, efficiency of probe cleavage, purity of the probe, and setting of the fluorescence threshold in the detection instrument utilized.

Therefore, the constant K does not have to be equal to one. Assuming efficiencies of the target and the reference are the same:

$${E}_{X} = {E}_{R} = E , \\ \frac{{X}_{O}}{{R}_{O}} {x ( 1 + E )}^{{C}_{T , X} - {C}_{T , R}} = K$$

$$or$$

$${X}_{n} \times {( 1 + E )}^{Δ {C}_{T}} = K$$

where
X_{N}=X_{O}/R_{O}, the normalized amount of target
^{ΔC}^{_{T}}=C_{T,X}−C_{T,R}, the difference in threshold cycles for target and reference.

Rearranging gives the following expression:

X_{N}=K×(1+E)^{-ΔC}^{_{T}}

X_{N} for any sample q can be divided by the X_{N} for the calibrator (cb), yielding the following relationship:

$$\frac{{X}_{N , q}}{{X}_{N , cb}} = \frac{K {x ( 1 + E )}^{- Δ {C}_{T , q}}}{K {x ( 1 + E )}^{- Δ {C}_{T , cb}}} = {( 1 + E )}^{- ΔΔ {C}_{T}}$$

$$where$$

$$ΔΔ {C}_{T} = Δ {C}_{T , q} - Δ {C}_{T , cb}$$

For amplicons less then about 150 bp, the efficiency can be close to one. Therefore, the amount of target, normalized to an endogenous reference and relative to a calibrator can be given by:

2^{-ΔΔC}^{_{T}}

For the ΔΔC_{T} calculation to be valid, the efficiency of the target amplification and the efficiency of the reference amplification must be approximately equal. Before using the ΔΔC_{T} method, a validation experiment can be performed to demonstrate that the efficiencies of the target and reference are approximately equal. If this is demonstrated, the ΔΔC_{T} calculation for relative quantification of target can be used without running standard curves. If efficiencies of the target and the reference are not equal, quantification can be performed using the standard curve method. Alternatively, new primers can be designed and synthesized so that the target and reference are approximately equal so that the ΔΔC_{T} calculation may be used. When determining the gene copy number, the formula can be:

2×2^{-ΔΔC}^{_{T}}

Various embodiments of the present teachings can include methods of determining a copy number of the target gene in a sample genome. These methods can comprise forming a reaction mixture comprising: a sample comprising the sample genome; a target gene primer pair, a target probe comprising a first fluorophore and a minor groove binder, a reference gene primer pair, a reference gene probe comprising a second fluorophore and a minor groove binder, a DNA polymerase, then amplifying the target gene and the reference gene, determining threshold cycle values for the target and the reference; and determining a difference between the cycle threshold values of the target gene and the reference gene.

In various embodiments, a SNP genotyping assay can comprise a single tube comprising two primers that are complementary to genomic sequences flanking a SNP, and two probes, a probe complementary to a first SNP allele and a probe complementary to a second SNP allele. In certain embodiments, each probe comprises a fluorophore, and a fluorescence quencher, such as a non-fluorescent fluorescence quencher (NFQ). In some configurations, the fluorophore can be comprised by the 5′ end of the probe, and the fluorescence quencher can be comprised by the 3′ end of the probe. In some embodiments, the probe can further comprise a minor groove binder (MGB), and in some configurations, an MGB and an NFQ can be combined in the same moiety. A combined MGB and NFQ can be, for example, an MGB and NFQ supplied as an Eclipse™ MGB by Epoch Biosciences, Bothell Wash. In various embodiments, the probes comprise different fluorophores, for example a VIC dye can be linked to the 5′ end of a probe for a first allele and a FAM dye can be linked to the 5′ end of a probe for a second allele.

Without being limited by theory, it is believed that incorporation of an MGB into a probe increases the melting temperature (Tₘ) of a probe/target duplex, without increasing probe length. It is further believed that the presence of an MGB in a probe can result in greater differences in Tₘ values between matched and mismatched probes, thereby resulting in more accurate allelic discrimination.

Various fluorophores can be used in a such as, for example, FAM, VIC, SYBRA Green 1 HEX, JOE, ROX, ALEXA, TEXAS RED, Commercially available filters for fluorophores include FAM™/SYBR® Green I, TET, HEX™/JOE™/VIC™, TAMRA™, Texas Red®/ROX™ Cy7™, Cy5™, Cy3™, and ALEXA Fluor® 350 filter sets. These fluorophores and filters are well known in the art and are available through a variety of sources such as Applied Biosystems, Foster City, Calif., Stratagene, San Diego, Calif., Qiagen, Inc. Valencia, Calif.; and Promega, Madison, Wis.; Molecular Probes, Inc., Eugene Oreg.; and Chroma Technology Corp., Rockingham, Vt.

In various embodiments of the present teachings, a fluorogenic assay can utilize probes such as, for example Taq Man® (Applied Biosystems, Inc.) probes, as well as primers that are complementary to genomic sequences flanking a genetic locus. A TaqMan probe can comprise a sequence complementary to an allele, as well as a fluorophore and a fluorescence quencher.

FIG. 1 is an illustration of data describing discrimination of alleles for SNP using a VIC-NFQ-MGB and FMA-NFQ-MGB probes on genomic DNA samples from human individuals. The data illustrate that a mismatch can have a disruptive effect on hybridization.

In various embodiments, a target gene can be a cytochrome gene. In certain embodiments of the present teachings, the cytochrome gene can be a cytochrome P450 gene, selected from the group consisting of CYP1A1, CYP1A2, CYP2A1, CYP2A6, CYP2A7, CYP2B6, CYP2C8, CYP2C9, CYP2C19, CYP2D6, CYP2E1, CYP3A4, CYP3A5, CYP3A7, CYP4B1, CYP5A1, CYP8A1, and CYP21. In other embodiments, the target gene can be selected from the group consisting of NAT1, NAT2, COMT, TMPT, and TYMS. In various embodiments of the present teachings, the target gene can be selected from the group consisting of a constitutive androstane receptor, a pregnane X receptor, an alcohol dehydrogenase, a flavin monooxygenase, a glutathione S-transferase, a transporter, an oATP-C, an epoxide hydrolase, a carboxylesterase, a monoamine oxidase, a paraoxonase, sulfotransferase, and a UDP-glucuronosyl-transferase. In certain embodiments, the alcohol dehydrogenase can be selected from the group consisting of ADH1A, ADH1B, ADH1C, ADH4, ADH5, ADH6, and ADH7. In other embodiments, the monooxygenase can be selected from the group consisting of FM01, FM03, FM04, and FM05. In some embodiments, the glutathione S-transferase can be selected from the group consisting of GSTM1 and GSTT1. In other embodiments, the transporter can be selected from the group consisting of MDR1, MRP1, MRP2, and MXR. Various embodiments of the present teachings include methods for analyzing drug metabolizing enzymes.

### Example 1

Gene deletions, as well as single nucleotide polymorphisms (SNPs), in the cytochrome P450 and glutathione S-transferase families have been associated with variation in drug metabolism and with certain cancers. The following is a non-limiting example of a method of the present teachings to determine the copy number (0, 1, or 2) of CYP2D6, GSTM1 and GSTT1 genes in a population of individuals. Real-time quantitative PCR simultaneously measures the threshold cycle values of the target gene amplification (CYP2D6, GSTM1, or GSTT1) and the reference gene amplification (RNaseP, which is present in one copy per haploid genome). ΔCT can be determined by subtracting the reference CT from the target CT, and relative quantity can be calculated by 2⁻²^{_{T}}^{-calibrator C}^{_{T}}. This 2^{-ΔΔC}^{_{T}} formula is described in detail above. The copy number, or “gene dosage”, can be the product of 2 and the relative quantity.

The gene dosage method takes advantage of a TaqMan® assay using a sequence detection system such as the ABI PRISM 7700® instrument with Sequence Detection Software (SDS), available from Applied Biosystems, Foster City, Calif. Gene dosage uses the relative quantification analysis, in which the PCR signal of the target gene is detected by the FAM-labeled TaqMan probe and RNase P Gene is detected by the VIC-labeled probe. Target genes (CYP2D6, GSTM1 or GSTT1) are normalized by the reference gene (RNaseP). Thirty-one genomic DNA (gDNAs) were assayed, in triplicate, per target gene. The amount of target, normalized to the reference and relative to a calibrator (in this case, an arbitrary individual with two copies of genes), is calculated using the 2^{-ΔΔC}^{_{T}} method. This value is multiplied by 2 to determine gene copy number.

In these experiments, genomic DNA from individuals from a variety of ethnic groups (Caucasian, Chinese, Indo-Pakistani, African American, Middle Eastern, SW American Indian, Japanese, Mexican, Puerto Rican) was obtained from Coriell Cell Repositories, Camden, N.J.

These experiments also used 20× Primer Probe Mixes, 4 μM FAM or VIC probe+2 μM each primer, all provided by Applied Biosystems. Reaction mixtures were comprised as follows:

| | |
|---|---|
| 2X TaqMan ® Universal Master Mix | 25 μl |
| 20X FAM Target Primer Probe Mix | 2.5 μl |
| 20X VIC RNase P Primer Probe Mix | 2.5 μl |
| gDNA (4 ng/μl) | 5 μl |
| Sterile Water | 15 μl |
| Total Volume | 50 μl |

PCR conditions were as follows:

| | | |
|---|---|---|
| 50° C. | 2 | minutes HOLD |
| 95° C. | 10 | minutes HOLD |
| 92° C. | 15 | seconds |
| 60° C. | 1 | minute |
| Laser exposure time | 25 | msec |

Primers and Probes for amplification of the following human loci were used for human gene loci CYP2D6 and GSTM1.

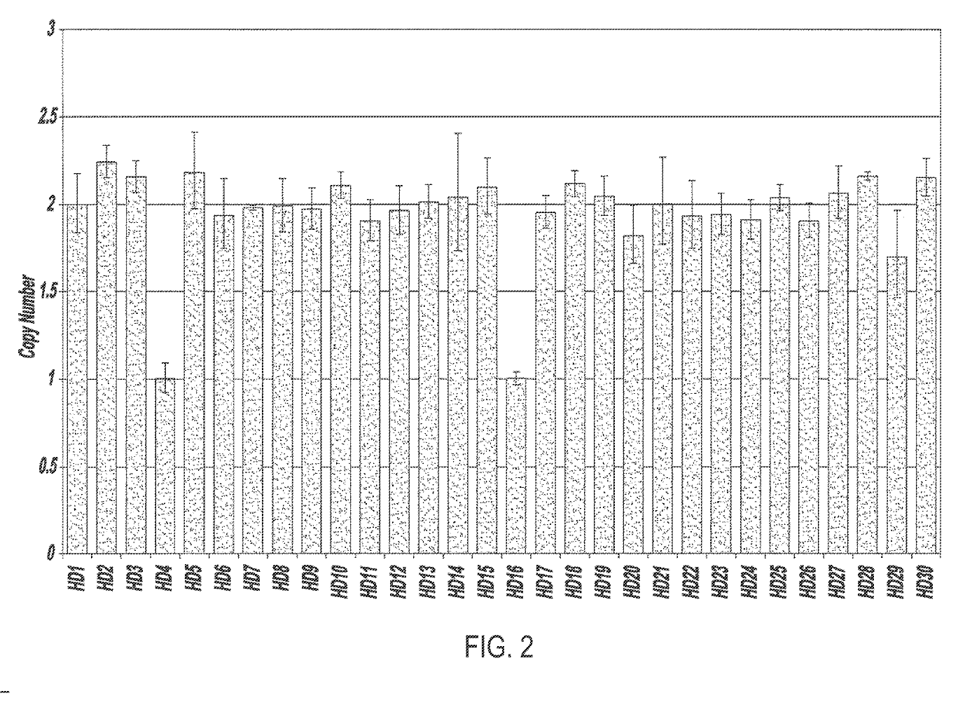

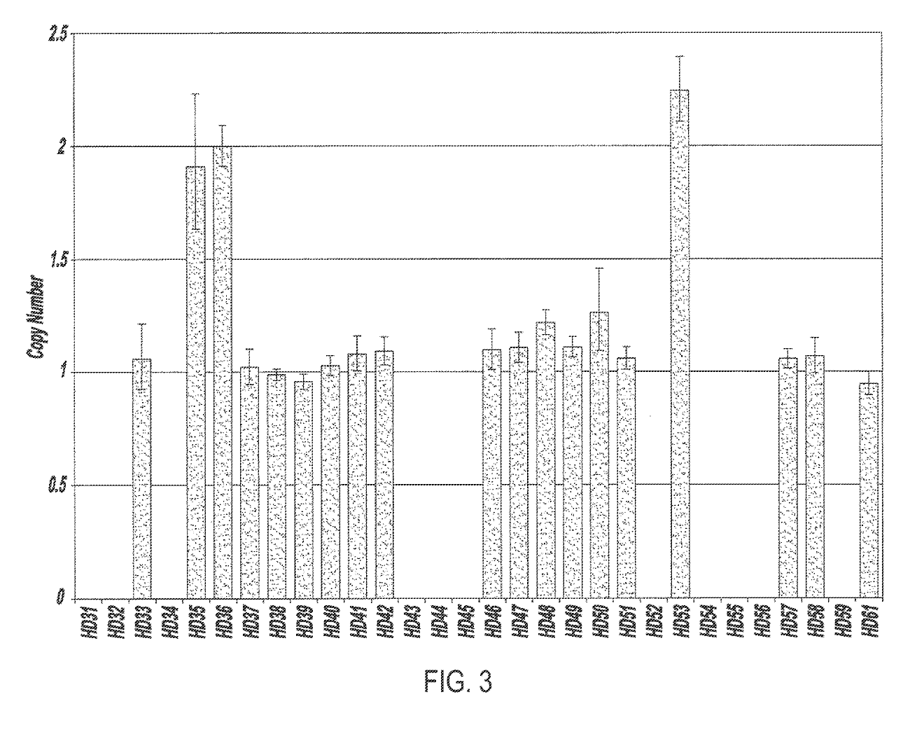

Results are shown in FIGS. 2 and 3. FIG. 2 shows that there is unambiguous discrimination of 2 individuals with one copy of CYP2D6 and 28 individuals with 2 copies. Maximum error bars (Std+) were determined by (Copy Number−Std 1), where Std 1=(2−((Average Ct−Standard Deviation)−Calibrator Ct))*2. Minimum error bars (Std−) were determined by (Std 2−Copy Number), where Std 2=(2−((Average C_{T}+Standard Deviation)−Calibrator C_{T}))*2. FIG. 3 shows that there is a clear discrimination of individuals with 0, 1, or 2 copies of the GSTM1 gene. Eleven subjects do not have this gene, 16 have 1 copy, and 3 have 2 copies. Maximum and minimum error bars were calculated as described above.

### Example 2

This example illustrates a method for detecting a candidate genetic anomaly in a population. First, a fluorogenic SNP genotyping an assay is performed using TaqMan® probes and primers and the data is displayed on a graphical interface, resulting in a data readout similar to that shown in FIG. 4, upper left panel. In this display, there are clusters representing individuals homozygous for each SNP allele, as well as a cluster representing individuals heterozygous for both SNP alleles. In addition, there are two groups of data points (circled) which do not fall within any of the clusters. These data points represent individuals whose genomes comprise a candidate genetic anomaly such as a candidate genetic duplication.

### Example 3

This example illustrates a method for quantifying sequence copy number in individual genomes comprising a candidate genetic anomaly. In this example, genomes of individuals represented in the data of Example 2 comprising a genetic anomaly were analyzed for gene copy number. Real time quantitative PCR was used to measure the threshold cycle values of a target gene amplification (CYP2E1) and a reference gene amplification (RnaseP, which is present in one copy per haploid genome). Data was analyzed using a 2^{-ΔΔC}^{_{T}} method as described supra.

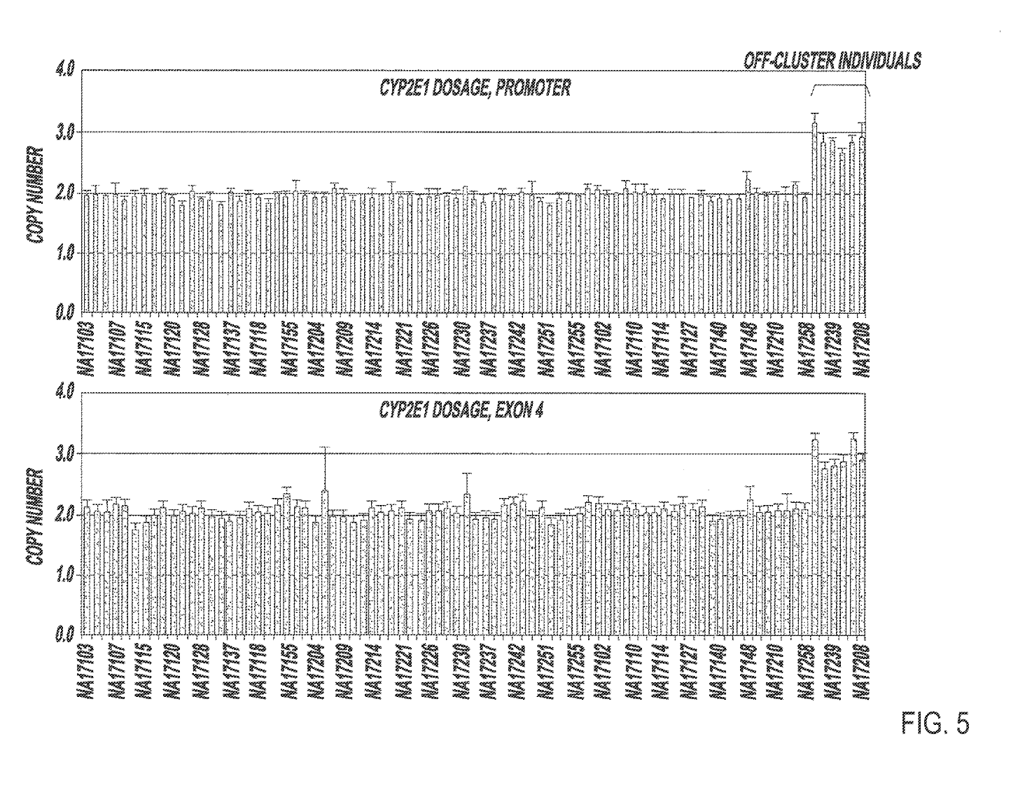

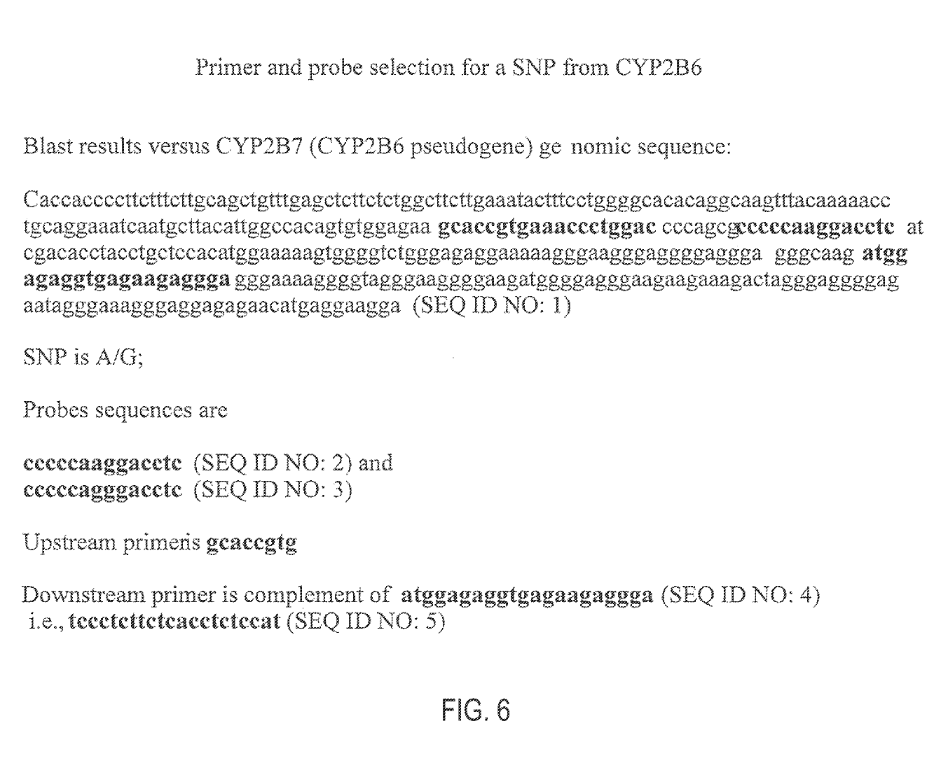

As shown in FIG. 5, there is unambiguous discrimination and identification of 6 individuals comprising 3 copies of the CYP2E1 gene and 22 individuals comprising 2 copies of the CYP2E1 gene.

### Example 4

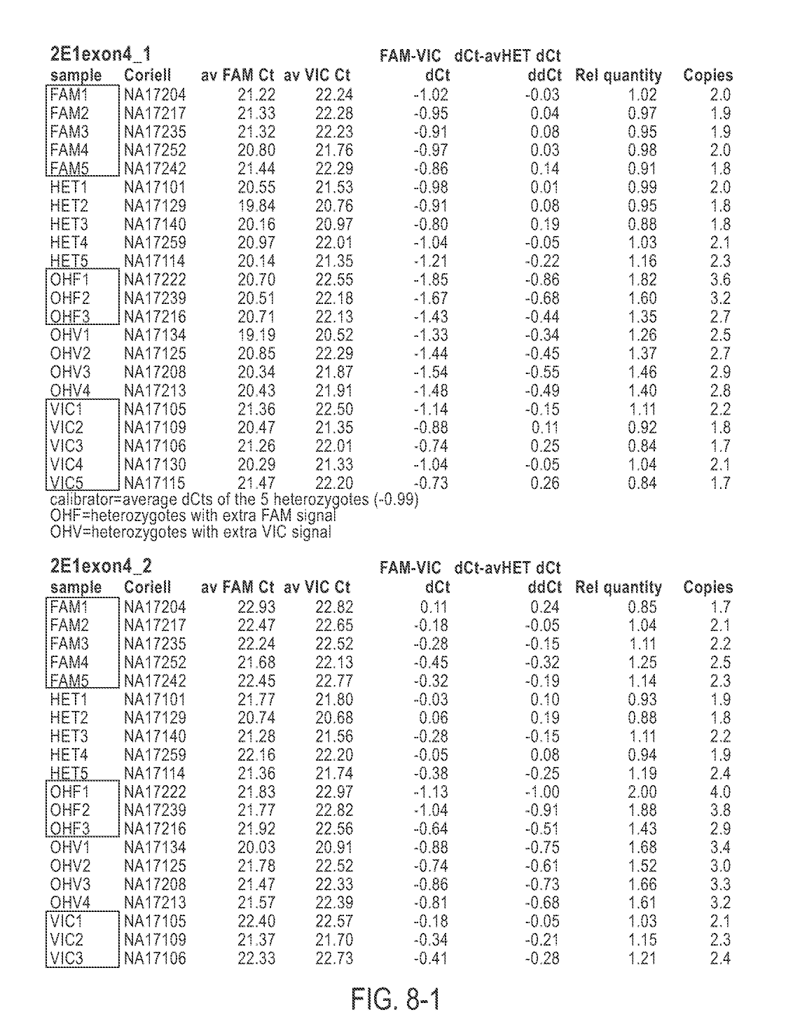

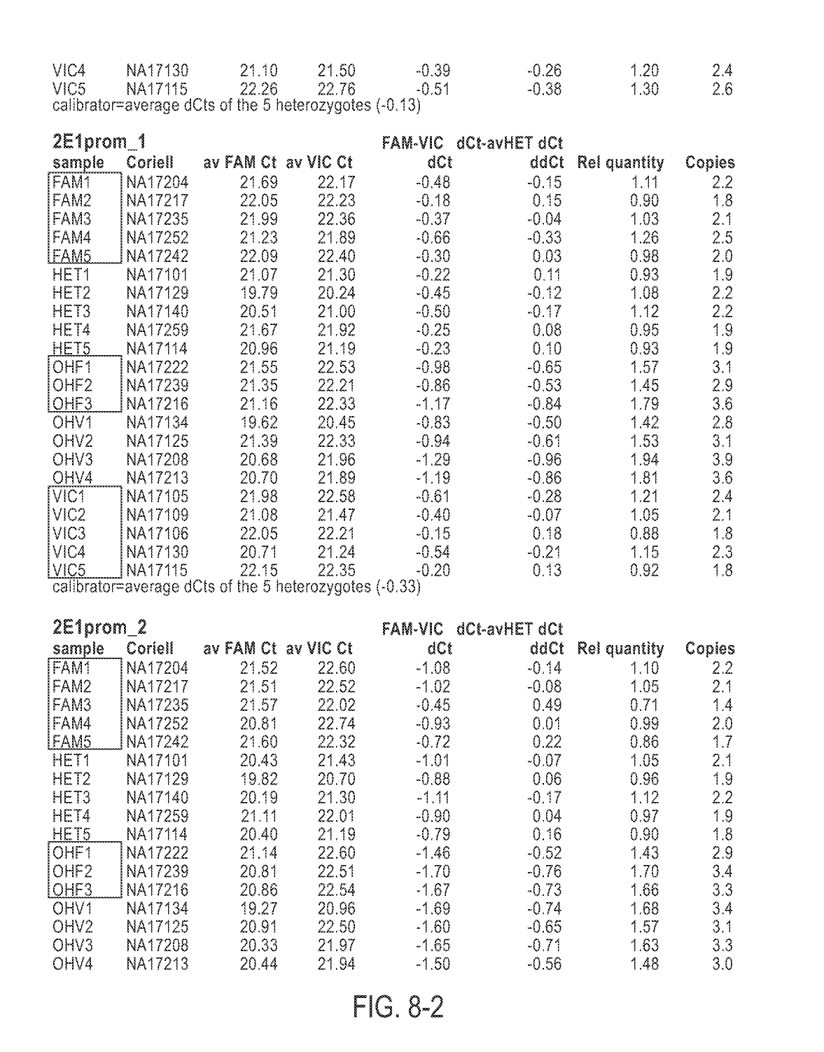

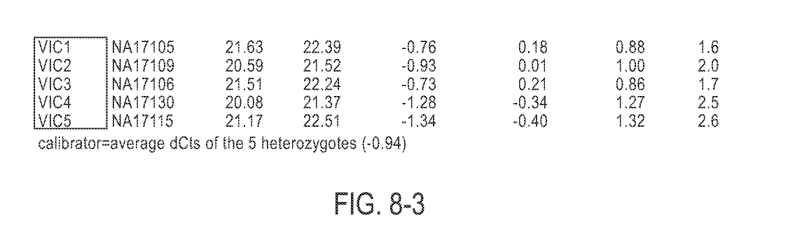

This example illustrates results for four gene dosage assays to CYP2E1. In this example, as shown in FIG. 8, four non-overlapping primer/probe sets were used to measure gene dosage in 7 samples previously identified as falling into extra clusters and 15 other DNA samples that typed as either homozygotes or heterozygotes with the E_CYP2E1*7W-10 SNP assay. On both the 7700 and 7900HT, those samples that fell into the extra clusters were quantified as having three copies of the CYP2E1 gene, and the samples that fell into the expected three clusters were quantified as having two copoeis of the CYP2E1 gene. Thus, the samples that fell “off-cluster” in the SNP genotyping assay have an extra copy of the gene compared to the samples that genotype in one of the three expected clusters. The actual number of copies in any individual can be the whole number nearest the calculated number of copies shown in the right-most column.

As various changes could be made in the above methods and compositions without departing from the scope of the present teachings, it is intended that all matter contained in the above description be interpreted as illustrative and not in a limiting sense. Unless explicitly stated to recite activities that have been done (i.e., using the past tense), illustrations and examples are not intended to be a representation that given embodiments of this present teachings have, or have not, been performed.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinency of the cited references.

1-24. (canceled)

25. A system for identifying a candidate genetic anomaly in a population, the system comprising a graphical interface which exhibits a plurality of data points, wherein each data point occupies a position representing fluorescence intensities of a first fluorophore and a second fluorophore from an individual genomic sample subjected to a fluorogenic assay for alleles of a genetic locus, wherein fluorescence intensity of a first fluorophore is indicative of the presence of a first allele and fluorescence intensity of a second fluorophore is indicative of the presence of a second allele, and wherein a cluster of data points is indicative of a genome homozygous for the first allele, a genome homozygous for the second allele, or a genome heterozygous for the first and second alleles, and wherein a data point outside any cluster represents an individual genome comprising a candidate genetic anomaly.

26. A system in accordance with claim 25, wherein a fluorogenic assay comprises:
forming a mixture comprising a genome of an individual, a first nucleobase primer comprising a sequence which maps upstream from the genetic locus, a second primer complementary to a sequence which maps downstream from the genetic locus, a first probe comprising the first fluorophore and a nucleic acid sequence complementary to a genomic sequence comprising the first allele, and a second probe comprising the second fluorophore and a nucleic acid sequence complementary to a genomic sequence comprising the second allele, and a thermostable DNA polymerase having 5′ exonuclease activity;
subjecting the mixture to thermal cycling; and
detecting fluorescence intensities of each of the first fluorophore and the second fluorophore.

27. A system in accordance with claim 26, wherein the detecting fluorescence intensity comprises end point detection of fluorescence intensity.

28. A system in accordance with claim 26, wherein the mixture further comprises a control fluorophore, and the detecting fluorescence intensity comprises detecting fluorescence intensity normalized to a control fluorophore fluorescence intensity.

29. A system in accordance with claim 28, wherein each cluster of data points comprises a cluster of fluorescence intensities normalized to the control fluorophore fluorescence intensity, and wherein data points outside of any cluster are data points indicating the presence of a gene duplication.

30. A system in accordance with claim 29, wherein each cluster of data points comprises a cluster of fluorescence intensities normalized to the control fluorophore fluorescence intensity, and wherein data points outside of any cluster are data points indicating the presence of a candidate gene duplication.

31. A system in accordance with claim 28, wherein each cluster of data points comprises a cluster of fluorescence intensities normalized to the control fluorophore fluorescence intensity, and wherein data points outside of any cluster are data points indicating the presence of a candidate third allele.

32. A system in accordance with claim 28, wherein each cluster of data points comprises a cluster of fluorescence intensities normalized to the control fluorophore fluorescence intensity, and wherein data points outside of any cluster are data points indicating the presence of a candidate non-specific primer.

33. A system in accordance with claim 28, wherein each cluster of data points comprises a cluster of fluorescence intensities normalized to the control fluorophore fluorescence intensity, and wherein data points outside of any cluster are data points indicating the presence of a candidate allele under a primer.

34. A system in accordance with claim 28, wherein each cluster of data points comprises a cluster of fluorescence intensities normalized to the control fluorophore fluorescence intensity, and wherein data points outside of any cluster are data points indicating the presence of a candidate allele under a probe.

35. A system in accordance with claim 25, wherein the first fluorophore and the second fluorophore are different and are each selected from the group consisting of FAM, VIC, Sybra Green, TET, HEX, JOE, NED, LIZ, TAMRA, ROX, ALEXA, Texas Red, Cy3, Cy5, Cy7, Cy9, and dR6G.

36. A system in accordance with claim 25, wherein the first fluorophore and the second fluorophore are selected from FAM and VIC.

37. A system in accordance with claim 28, wherein the control fluorophore is different from the first fluorophore and the second fluorophore, and is selected from the group consisting of FAM, VIC, Sybra Green, TET, HEX, JOE, NED, LIZ, TAMRA, ROX, ALEXA, Texas Red, Cy3, Cy5, Cy7, Cy9, and dR6G.

38. A system in accordance with claim 25, wherein a cluster comprises at least two data points.

39. A system in accordance with claim 25, wherein the graphical interface further comprises a scatterplot displayed on coordinate axes.

40. A system in accordance with claim 39, wherein coordinate axes are orthogonal coordinate axes.

41. A system in accordance with claim 25, wherein the graphical interface is comprised by a digital computer monitor.

42. A system in accordance with claim 25, wherein the alleles of the genetic locus are SNP alleles.

43-72. (canceled)


SEQUENCE LISTING (6 sequences)

SEQ ID NO: 1 - DNA, 360 bp, human
caccaccccttctttcttgcagctgtttgagctcttctctggcttcttgaaatactttcctggggcacacaggcaagtttacaaaaacctgcaggaaatcaatgcttacattggccacagtgtggagaagcaccgtgaaaccctggaccccagcgcccccaaggacctcatcgacacctacctgctccacatggaaaaagtggggtctgggagaggaaaaagggaagggaggggagggagggcaagatggagaggtgagaagagggagggaaaaggggtagggaaggggaagatggggagggaagaagaaagactagggaggggagaatagggaaagggaggagagaacatgaggaagga

SEQ ID NO: 2 - DNA, 14 bp, Artificial
  snythetic probe
cccccaaggacctc

SEQ ID NO: 3 - DNA, 14 bp, Artificial
  synthetic probe
cccccagggacctc

SEQ ID NO: 4 - DNA, 21 bp, Artificial
  synthetic primer
atggagaggtgagaagaggga

SEQ ID NO: 5 - DNA, 21 bp, Artificial
  snythetic primer
tccctcttctcacctctccat

SEQ ID NO: 6 - DNA, 8 bp, Artificial
  snythetic primer
gcaccgtg